(19) United States
(12) Patent Application Publication
(10) Pub. No.: US 2017/0067889 A1
(43) Pub. Date: Mar. 9, 2017

(54) Lateral Flow Diagnostic Devices with Integrated Electronic Components and Methods of Use Thereof

(71) Applicant: Idan TAMIR, Zichron Ya'akov (IL)

(72) Inventor: Idan TAMIR, Zichron Ya'akov (IL)

(21) Appl. No.: 15/247,210

(22) Filed: Aug. 25, 2016

Related U.S. Application Data
(60) Provisional application No. 62/214,048, filed on Sep. 3, 2015.

(51) Int. Cl. G01N 33/543; G01N 27/414; G01N 27/327

(52) U.S. Cl. CPC G01N 33/54373; G01N 33/5438; G01N 27/3275; G01N 27/4145

(57) ABSTRACT

A biological sample device for detecting the presence or absence of specific analytes in a sample is provided. A sample, such as a blood or urine sample or a pre-processed tissue sample is collected on a porous pad. A first membrane in liquid communication with the sample pad includes an analyte-specific, electroactively-labeled detection reagent reactive with the an analyte. A second membrane in liquid communication with the first membrane includes a biosensor whose surface has been modified by an analyte-specific capture reagent. The analyte-binding capture reagent immobilizes the analyte electroactive label complex on the surface of the biosensor, whereby direct interaction of the electroactive label with the surface of the biosensor generates an electric signal used by an electronic processing unit to determine whether the analyte is present in the sample.

This application claims priority under 35 U.S.C. §119(e) to U.S. provisional application Ser. No. 62/214,048, filed Sep. 3, 2015, the entire disclosure of which is incorporated by this reference into the present U.S. patent application.

### BACKGROUND AND SUMMARY OF THE INVENTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Lateral flow devices have been extensively used for rapid testing and diagnosis of a variety of clinical and pathological conditions in both point-of-care (POC) and home settings. Although these lateral flow tests (LFTs) have many advantages, such as being based on a mature technology that enjoys wide user acceptance and recognition, clear development & regulatory paths, established manufacturing processes, and is both cost-effective and scalable, they have several disadvantages that limit their wide-spread use, especially for self-testing. These disadvantages include, among other: challenging test performance protocols, especially in the hands of untrained individuals; compromised and user-dependent test accuracy and reproducibility; ambiguity in test results detection—LFTs are mostly qualitative in nature and depend on visual signal detection for their interpretation; problematic results interpretation and correlation—consumers are often confused about what to do with RDT results and how these would correlate with test results obtained at the POC or hospital lab. Additionally, results traceability and their communication to the healthcare professional are challenging with current RDTs.

Recently, advances in optical reader device technologies, including reduced optical components cost and their miniaturization have significantly improved LFT readout accuracy, also promoting the transition of LFT from qualitative to quantitative tests and transfer of such readers from POC to patients' homes for self-testing. Among such technological advancements, most notable are LFT-integrated optical readers, such as in the Clearblue pregnancy test, and the use of mobile phones (together with the required additional hardware and software) for LFT signal detection and quantification.

A different approach to devices for biological and clinical sample diagnosis combines microfluidics technologies for sample processing with electronic biosensors specific for individual analytes. Such devices, generally referred to as “Lab-On-Chip” (LOC), have gained wide popularity and acceptance in hospital laboratory settings and POCs, partially replacing traditional and high-throughput automatic diagnostic platforms. Although these LOC devices provide accurate diagnostic test results for multiple analytes in a relatively short period of time, their cost and the significant user expertise required for their proper operation has generally rendered them unsuitable for testing outside the POC.

The field of electronic biosensors is well-developed with many examples of their successful integration into diagnostic products (e.g., blood glucose monitoring). Amperometric biosensors generally rely on electrochemical reactions mediated by specific enzymes (e.g., glucose oxidase) for analyte detection and quantification, and are less optimal for detecting and quantifying binding events at the biosensor surface. Potentiometric biosensors, such as Field-Effect Transistors (FET), have the capability of detecting molecular binding events due to their effect on the electric field sensed by the biosensor's channel/gate, which influences source-drain current. However, non-specific interactions between the channel's surface material and the myriad of biochemical entities present in biological samples may overwhelm specific interactions with the analyte of choice, limiting FETs signal-to-noise ratio (S/N) and rendering them sub-optimal for clinical diagnostic applications. In order to minimize such non-specific interactions, potentiometric biosensor-based POC devices (e.g., iSTAT by Abbott) rely on elaborate and relatively expensive microfluidic technologies, limiting their application to professional, LOC-based systems.

A relatively new development in the electronic biosensor area is the ability to directly detect analyte binding to a sensor electrode employing techniques such as electrochemical impedance spectroscopy (EIS). EIS potentially enables highly-sensitive, label-free detection of biomolecular binding events at the electrode's surface by measuring frequency-dependent changes in electrical impedance. Although EIS has been extensively investigated in the laboratory, it has so far gained limited utilization in POC diagnostic applications and products, putatively due to its intrinsic low signal-to-noise ratio (S/N) in detecting analytes in biological samples, combined with requirement for sophisticated and expensive instrumentation for signal detection and analysis.

Taken together, the above limitations have until now restricted the use of the different electronic sensing technologies in diagnostic applications where target recognition is achieved by detecting its specific interaction with an immobilized capture reagent. Furthermore, implementing these different electronic sensing modalities within the framework of disposable lateral flow tests raises additional cost and performance issues that have until now hampered the adoption of such diagnostic devices. One of the main issues to address when attempting the utilization of these and other biosensor technologies is their S/N, which typically limits their analyte cutoff value (also known as limit of detection (LOD)) to the nM range. Cutoff improvement has been addressed by different approaches, including: i) increasing the surface area of the sensing electrode/gate by different modifications (e.g., depositing gold nanoparticles (GNP), carbon nanotubes (CNT) and other conducting entities on the biosensor surface); ii) employing enzyme-mediated amplification schemes to generate species that diffuse to the electrode surface and become involved in a redox reaction (e.g., alkaline phosphatase, used in the iSTAT cardiac troponin I (cTnI) diagnostic device to generate a redox species); iii) reducing the molecular distance between analyte biding site and the biosensor electrode surface—especially for EIS and FET applications where the effect of such binding on the electric field and/or current sensed by the gate/electrode is exponentially dependent on such distance. Such distance reduction was attempted using fragments of the analyte-capturing entity (e.g., Fab fragments of antibodies) or shorter analogs thereof (e.g., aptamers); iv) using electroactive labels (e.g., gold nanoparticles (GNP), carbon nanotubes (CNT), etc.) to enhance the signal generated by the individual binding event, by binding such entities to the detection reagent used in ELISA detection schemes.

It is important to note that the above-mentioned approaches for LOD improvement had, as of yet, limited impact on the wide-spread adoption of such devices, with the exception of the mentioned iSTAT amperometric technology. EIS and FET technologies are still lagging behind in their utilization for commercial biosensor fabrication.

The present invention employs electroactive labels in combination with different strategies aimed at bringing such labels in close proximity to the sensing electrode surface. Such close proximity between the electroactive label and electrode surface maximizes the former's signal enhancement effect on specific analyte binding events. This approach is a variation of the “electrochemical ELISA” concept, where the analyte-specific conjugate used to label the analyte, allowing its electrochemical detection at the biosensor's capture zone, is being modified to allow its direct interaction with the biosensor surface. This unique approach combines employing high-affinity, specific interactions between the analyte and both the labeling and capture reagents, with lower-affinity interactions between the label and electrode surface that bring the latter two in close proximity. It is important to note that such lower-affinity interactions should be carefully engineered in order to minimize non-specific binding of the electroactive label to the electrode surface in the absence of the higher-affinity interactions between the analyte and both the labeling and capture reagents.

The device of the present invention also combines the advantages of LFT with those of electronic biosensors in an electronic lateral flow test (ELFT) format, thereby overcoming some of the major barriers for usage of such devices by untrained individuals and in settings where access to electricity and/or sophisticated instrumentation (such as dedicated readers) is limited or non-existent. Additionally, this unique ELFT platform allows detecting targets with increased sensitivity and specificity, without the need for visual or electro-optical sensors for results reading. This is achieved using the above-mentioned novel analyte labeling and detection schemes, combined with signal gating and acquisition protocols that take advantage of the passive capillary flow of reagents (including analyte) along the membrane that is part of the lateral flow component of this device. Transferring LFT signal detection mode from the traditional optical one into electronic has several advantages, such as user-independent and therefore unbiased test performance that should significantly reduce user-originated errors. Such reduced user dependence in test performance and results reading is expected to consequently also reduce false positive and false negative rates, thereby increasing test accuracy and reliability.

Overcoming noise originating from non-specific binding events while amplifying specific ones is key to successful implementation of EIS- or FET-based biosensors in a diagnostic device. The device of the present invention makes use of several procedures and protocols, either individually or in different combinations, to achieve this goal in an ELFT format. Among these are:
i. Use of passive and active membrane components to filter and/or bind interfering substances
ii. Use of electroactive materials as markers for in-situ analyte labeling, thereby differentiating such analyte from non-specific counterparts. Such electroactive labels serve to amplify the initial electric signal resulting from specific analyte binding to the biosensor surface
iii. Combining high-affinity and specific analyte binding to labeling and capture reagents together with engineered low-affinity interactions between the electroactive label and the biosensor surface, serves to further enhance the effect of analyte binding to the biosensor surface on electrical signal
iv. Use of interdigitated, individually-addressable, in-line control electrodes to detect non-specific binding events contribution to the overall change in biosensor electric signal
v. Use of electronic gating, time-dependent signal acquisition and electronic filtering protocols to further reduce the level of biosensor signal noise

Targeting the device of the present invention for use in point-of-care and home settings entails a significant cost sensitivity requirement. In order to achieve such cost saving goal, the device of the present invention optionally employs both disposable and reusable components. The reusable component contains a central processing unit (CPU) that is electrically-connected by detachable leads to the disposable LFT component that carries the biosensor integrated with the lateral flow test membrane and reagents. This novel design spares the CPU component for the performance of additional tests, while the rest of the device (disposable LFT component/cartridge) is being disposed-of following single use. The detached CPU can then be reconnected through its leads to a new disposable LFT cartridge for the purpose of new test performance. The detached CPU can also be connected through a USB port or by wireless communication to a personal computer or smart phone for results display, or combined with an integral liquid crystal display (LCD) that allows direct results visualization without the need for computer or smart phone connection. Further CPU features include: data storage capability, disposable LFT cartridge attachment detection and identification, self-calibration, etc. It should be noted however that the reusability of the CPU is just one preferred embodiment of the present invention and a fully-disposable (i.e., including CPU) device is also within the scope of the current invention.

In some embodiments, the device of the present invention is configured to measure the presence or absence of specific analytes in a biological sample. In some embodiments, the device comprises: (i) a sample pad, for use to directly collect a clinical sample (e.g., urine) or a pre-processed sample, (ii) a lateral flow membrane, for use in sample fractionation and analyte transfer to the device's biosensor, (iii) a labile analyte-specific, electroactively-labeled detection reagent that is incorporated within the conjugate pad of the lateral flow device, (iv) a biosensor embedded at the detection zone within such lateral flow device, serving to specifically detect analytes and produce an electric signal in response to analyte binding that is being amplified by direct interaction between the electroactive label and the biosensor surface, (v) an electronic processing unit serving to process the biosensor signal, transforming it into digital output, (vi) a display unit serving to visually display the diagnostic digital output of the processing unit.

In some embodiments, the method of the present invention is comprised of: (i) collecting a biological sample by the sample pad of the device; (ii) allowing the sample containing the analyte to passively fractionate along the lateral flow membrane, including filtration/separation of intact cells and cell debris, followed by solubilization and reaction of the carried analyte with the electroactively-labeled detection reagent in the conjugate pad and further flow of the formed complexes along the membrane, reaching the detection zone and interacting with the biosensor, thereby triggering signal acquisition; (iii) allowing the processing unit to process the signal generated by the electroactive label-amplified specific binding of analyte to the biosensor and provide a diagnostic readout; (iv) determining the presence, absence, amount or concentration of analyte in the clinical sample being analyzed according to the provided readout.

In some embodiments, the method of the present invention is comprised of: (i) manually collecting a biological sample, followed by sample extraction using an appropriate extraction buffer; (ii) transferring the extracted sample to the sample pad of the device of the present invention; (iii) allowing the extracted analyte solution to passively fractionate along the lateral flow membrane, solubilize and react with the electroactively-labeled detection reagent in the conjugate pad and to further flow along the membrane, reaching the detection zone, where the electroactively-labeled analyte complex specifically binds to the biosensor; (iv) allowing the processing unit to process the signal generated by the electroactive label-amplified binding of analyte to the biosensor and provide a diagnostic readout; (v) determining the presence, absence, amount or concentration of analyte in the clinical sample being analyzed according to the provided readout.

In some embodiments, the device of the present invention incorporates labile analyte-specific reagents, such as antibodies, DNA, aptamers, enzymes, receptors, etc., that are stably-labeled by electroactive markers. In some embodiments, such labile analyte-specific reagents are incorporated within a specific location (e.g., equivalent to what is known in the art as the “conjugate pad”) along the lateral flow membrane, preferably in between the sample pad and the analyte detection zone wherein the biosensor is embedded. Incorporation of such labile analyte-specific reagents can be either to a separate membrane (i.e., conjugate pad) that is in fluid contact with the lateral flow membrane, or within the lateral flow membrane itself. Reagent application to the membrane is achieved by liquid solution application techniques known in the art, such as spraying, spreading, stamping, dipping, etc., followed by solvent evaporation, leaving the labeled reagent physically adsorbed to the membrane. Multiple such labeled reagents can be applied, each targeting a specific analyte. During ELFT operation, the labeled analyte-specific reagent dissolves in the analyte solution or the buffer used as the LFT running/development media, and specifically reacts with its target analyte. Formed electroactively-labeled analyte complexes migrate by capillary flow forces along the LFT membrane until they reach the biosensor and bind to the specific capture reagent immobilized on the electrode's surface.

In some embodiments, the lateral flow membrane of the present invention incorporates specific elements, positioned between the sample and conjugate pads, whose purpose is to filter cells and cell debris, bind potentially-contaminating materials, or otherwise separate sample components that interfere with optimal test performance. An example for such separation elements are membranes having asymmetric pore size that efficiently sieve blood cells, selectively allowing passage of soluble plasma components.

In some embodiments, the electroactive label described in the present invention is a molecule (e.g., polymer) or nanoparticle (e.g., noble metal nanoparticle), that are either: (i) conductive (e.g., gold nanoparticles (GNP) and carbon nanotubes (CNT)), (ii) exhibit a dielectric constant(s) different than the media surrounding the biosensor element, (iii) electrochemically reactive (e.g., may become involved in a redox reaction), (iv) catalyze the generation of electrochemically reactive species, or (v) highly-charged, e.g. polyelectrolytes.

In some embodiments, the device of the present invention incorporates an electronic biosensor embedded within a lateral flow membrane, thereby allowing direct fluid contact between the analyte solution and the biosensor surfaces. In some embodiments, the biosensor may be amperometric, potentiometric or impedimetric, in which the sensing element (i.e., electrode) has been modified to carry analyte-specific capture reagent on its surface. In some embodiments, analyte-specific capture reagent includes: antibodies, antigens, DNA, aptamers, enzymes, receptors, and generally any molecule that has micro-molar or higher affinity towards the target analyte.

In some embodiments, the biosensor incorporated in the device of the present invention is disposable and its sensing element is composed of an electrode array in which the electrodes are arranged parallel to each other and vertical to the direction of fluid flow through the lateral flow membrane. In some embodiments, the electrode array consists of co-planar interdigitated electrodes. In some embodiments, each electrode in the array is 100 nm to 100 μm wide, 100 μm to 10 mm long and spaced 100 nm to 1 mm apart. In some embodiments, the electrode's cross-section is essentially rectangular with a height of 10 nm to 10 μm above its underlying substrate. In some embodiments, the electrode is porous, with pore sizes ranging 1-100 nm.

In some embodiments, the electrodes that are part of the biosensor of the device of the present invention are constructed from conductive materials, such as (but not limited to): gold, copper, platinum, graphite, glassy carbon and conductive polymers (e.g., polypyrrole). In some embodiments the substrate that underlies the electrode is electrically semiconducting, dielectric or insulating. Prior to biosensor construction, the surface of these electrodes is cleaned, employing techniques known in the art and involving a combination of: mechanical and electrochemical polishing, chemical stripping, plasma treatment and various washing steps, etc., in order to remove contaminants and optimally achieve nascent, molecularly flat and chemically clean surfaces allowing further electrode manipulation steps required for biosensor construction.

In some embodiments, the external, clean surface of the electrodes of the device of the present invention is modified by various chemical reagents and techniques known in the art to fabricate chemically-reactive end groups—such as: maleimide, epoxy, and N-hydroxysuccinimide (NETS) ester—on such electrode surfaces. In some preferred embodiments, where the electrode's surface material is gold, the nascent electrode surface produced following surface cleaning is protected from undesired subsequent adsorption of contaminating chemical species by forming a densely-packed self-assembled monolayer (SAM) on its surface immediately following surface cleaning. Examples of appropriate SAM-forming substances are, but not limited to, organic sulfur-containing compounds that avidly bind to the gold surface via their sulfur atoms, such as: organic thiols, sulfides, thioesters and disulfides.

In some embodiments, the chemically-reactive end groups that are generated on the external surface of the biosensor electrode of the present invention are further reacted with an analyte-specific capture reagent, thereby covalently immobilizing such capture reagent to the electrode's external surface. Alternatively, the analyte-specific capture reagent may be directly immobilized to the biosensor surface through non-specific interactions, and/or specific linkers that are attached to the capture reagent and are reactive with the biosensor electrode's surface. An example for such specific linkers are bi-functional amine-reactive (e.g., NHS ester) organic molecules that incorporate a thiol functionality reactive with the gold electrode surface. Analyte binding to such electrode surface-immobilized capture reagent can be monitored by measuring the time-dependent electrochemical impedance signal (Z) between adjacent electrode pairs (working vs. reference electrode—Z_{wr}). In some embodiments of the present invention, Z_{wr} is compared with an equivalent signal—Z_{cr}—measured between a control electrode, carrying a reagent that is a close analog of the capture reagent but non-specific to the analyte (e.g., host species- and isotype-matched antibody), and the reference electrode. Both Z_{wr} and Z_{cr} signals emanate from electrode pairs that are positioned to equivalently interact with the analyte solution—i.e., they occupy equivalent positions with respect to fluid flow along the LFT membrane. Real-time difference between these two signals in terms of their phase and intensity (ΔZ(t)=Z_{wr}(t)−Z_{cr}(t) is then taken to represent the specific, time-dependent binding of the analyte to its capture reagent on the working electrode. Measurement of the time-dependent differential electrochemical impedance signal (ΔZ(t)) helps in minimizing non-specific contributions to the electrode's impedance from temporal and spatial variations in buffer composition, temperature, pH and biochemical composition of the tested sample.

In some preferred embodiments, the binding of the capture reagent to the working electrode's surface is mediated through electrically-conducting molecular structures, such as a conductive polymers (e.g., polypyrrole), carbon nanotubes (CNT), electrically-conducting self-assembling monolayers (ecSAM), or combination of ecSAM and gold nanoparticles (GNP), naming a few examples.

In some embodiments, the electronic processing unit (CPU) of the device of the present invention measures biosensor impedance signal, either under open circuit (zero current) or electrochemical conditions, by applying a sinus-modulated AC potential of 5-10 mV at several distinct frequencies in the range of 0.1 Hz to 10 MHz across the working (test) and reference electrodes. The same modulated AC potential is simultaneously applied between the control (see above for definition) and reference electrodes (see also FIG. 2B). The resultant output I_{wr} and I_{cr} currents are optionally mixed and integrated with the original AC signal to determine time-(t) and frequency (ω)-dependent changes in impedance between I_{wr} and I_{cr} (ΔZ(t,ω)). In case of measuring electrochemical impedance, a redox pair such as Fe(CN)₆^{-3/-4} is optionally incorporated in the buffer or the lateral flow membrane used for ELFT construction.

### BRIEF DESCRIPTION OF THE DRAWINGS

In order for the advantages of the invention to be readily understood, a more particular description of the disclosure briefly described above will be rendered by reference to specific embodiments that are illustrated in the appended drawing(s). It is noted that the drawings of the disclosure may not be to scale and are merely schematic representations, not intended to portray specific parameters of the disclosure. It is to be understood that these drawing(s) depict only typical embodiments of the disclosure and are not therefore to be considered to be limiting of its scope, the disclosure will be described and explained with additional specificity and detail through the use of the accompanying drawing(s), in which:

FIGS. 1A and 1B show top general views of an embodiment of the device of the present invention. FIG. 1A depicts externally-visible components, comprised of device cap and body, while FIG. 1B highlights some internal components, such as the electrode array, and the connection between the disposable and reusable parts of the device.

FIGS. 2A and 2B show in greater detail the fluidic and electrode array components of the device of FIGS. 1A and 1B. FIG. 2A depicts the positioning and integration of the electrode array in the device of the present invention. FIG. 2B shows typical individual electrode arrangement within the electrode array of the device.

FIGS. 3A-3C show several different approaches to electrode surface modification and capture antibody reagent immobilization to the activated electrode surface in accordance with the present invention.

FIGS. 4A and 4B show two embodiments of the electroactive-label impedance enhancement principle for analyte detection by EIS or FET in accordance with the present invention, employing electrode-immobilized analyte-specific antibodies in combination with electroactive marker-labeled soluble analyte-specific antibodies. FIG. 4A depicts a typical impedance-based sensor, where the specific binding of the electroactively-labeled detection antibody-analyte complex to the electrode-immobilized capture antibody brings the electroactive label in close proximity of electrode's surface thereby affecting electrode capacitance; FIG. 4B depicts a typical back-gated field effect transistor (FET), where the specific binding of the electroactively-labeled detection antibody-analyte complex to the gate-immobilized capture antibody-bound analyte brings the electroactive label in close proximity of the gate surface thereby affecting channel conductance.

FIGS. 5A-5C depict three different embodiments of electroactive label interaction augmentation modes with the biosensor surface in accordance with the present invention. FIG. 5A illustrates an electrostatic interaction between the electroactive label and the biosensor surface, mediated by engineering the electroactive label's zeta potential to be opposite of that of the biosensor surface. Such electrostatic interaction serves to both—attract the electroactive label to the biosensor surface and affect the biosensor surface charge distribution; FIG. 5B illustrates low-affinity binding of the electroactive label to the biosensor surface, mediated by engineering the biosensor surface to carry chemical groups that are either chemically-complementary to, or otherwise have low affinity to ones present in the electroactive label. FIG. 5C illustrates the use of an externally-applied electromangenic field to attract a captured electroactive and magnetic label to the biosensor surface.

### DETAILED DESCRIPTION

In some embodiments, the device of the present invention is configured to permit conducting an immunoassay for a specific analyte or set of analytes that are extracted from a biological sample or present in a biological fluid, such as blood, urine, saliva etc. In some embodiments, the device of the present invention integrates traditional lateral flow immunochromatographic assay technologies with those of electronic biosensors in a single apparatus, employing electroactive labels that act to amplify the change in impedance signal at a biosensor surface following detection reagent immobilization. Additionally, proper selection and construction of the electroactive label relative to the biosensor surface prevents non-specific contact between the two, promoting such interaction only upon the specific contact between the detection reagent, the analyte and the capture reagent. Such integration is expected to reduce the complexity of diagnostic test performance, while increasing test sensitivity and specificity, due in part to the non-biased nature of the employed signal detection protocols, since these won't rely on subjectively determining if a visually-discernible signal is formed on the lateral flow test strip. In some embodiments, the device of the present invention employs electroactive labeling techniques to enhance the difference in impedance signal following binding of a molecule to the surface of a biosensor electrode.

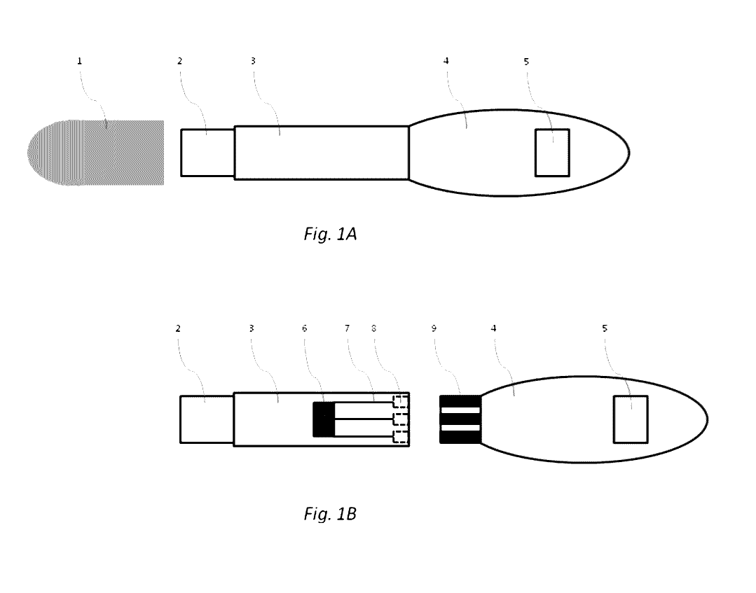

FIGS. 1A and 1B show top general views of an embodiment of the device of the present invention. FIG. 1A depicts externally-visible components, comprised of device cap (1), sample pad (2), electronic lateral flow component housing (3), central processing unit (CPU) housing (4) and a display (5). FIG. 1B highlights some of the device's internal components, such as the electrode array (6) that forms the biosensor, connected through insulated electric leads (7) to respective electric contact pads (8), allowing attachment and detachment of the CPU through matching electric contact pads (9) in its housing. It should be noted that although only three contact pads (8) and their matching counterparts (9) leading to the CPU are being depicted, additional contacts can be added and the electrode array may be expanded to include multiple repeats of the depicted array, or constructed of alternative electrode array arrangements. Such arrangements can be used, inter-alia, for assay multiplexing, detecting multiple analytes by a single electrode array. Together, elements 2, 3, 6, 7 and 8 form the disposable LFT component/cartridge referred to in the present invention, while 9, 4 and 5, together with the housed CPU itself, form the optionally-reusable component of the device of the present invention.

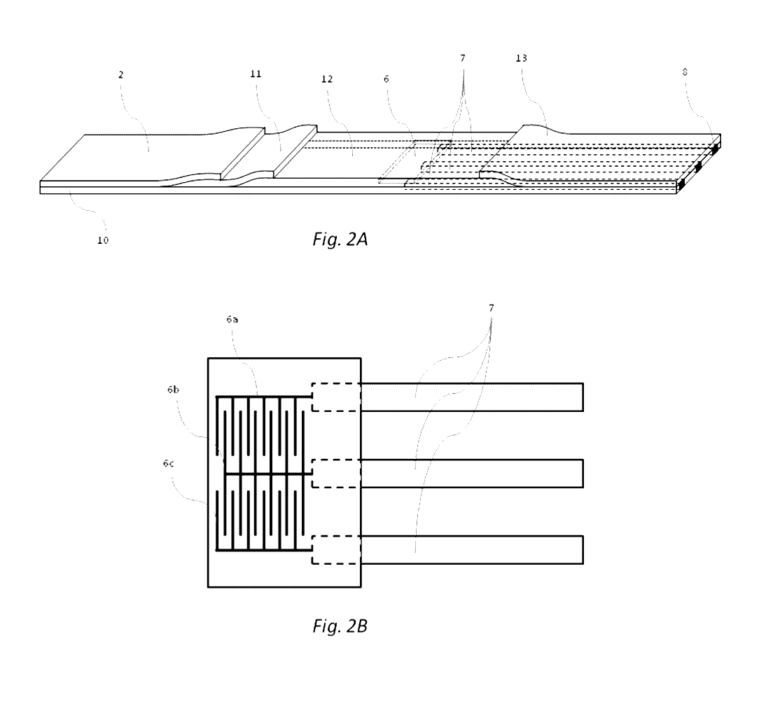

FIGS. 2A and 2B show in greater detail the fluidic and electrode array components of the device of FIGS. 1A and 1B. FIG. 2A depicts the positioning and integration of the electrode array, including the sample pad (2), the conjugate pad (11) which carries the electroactively-labeled detection reagent, the lateral flow membrane (12), supported by a backing material (10) to which the different membranes are laminated, the electrode array (6) and its insulated leads (7), ending in electrical contact pads (8), and a wicking pad (13) made of porous material that adsorbs the fluid that flows laterally away from the sample pad to the electrode array and from there to the wicking pad itself. FIG. 2B shows typical individual electrode arrangement within the electrode array of the device, where typically a working/test electrode (6a), carrying the immobilized analyte-specific capture reagent, is interdigitated with a reference/counter electrode (6b). As control serves an electrode (6c) situated equivalently to 6a with respect to the reference electrode and fluid flow. This electrode (6c) typically carries an immobilized control reagent (e.g., of the same host species and isotype as the analyte-specific capture antibody) in order to compensate for non-specific binding events and minimize inherent physiochemical differences between capture and control reagents. Impedance measurement is typically performed using electrode pairs 6(a-b) and 6(c-b) simultaneously and the resulting respective output currents—I_{wr} and I_{cr} are then processed by the CPU to determine time-(t) and frequency (ω)-dependent changes in impedance between I_{wr} and I_{cr} (ΔZ(t,ω)).

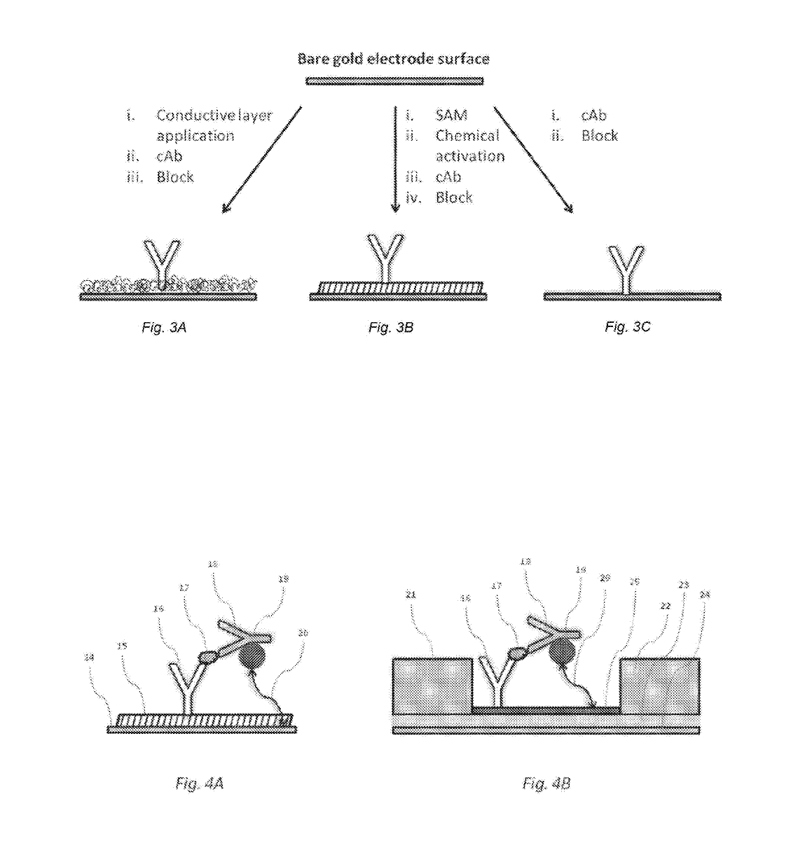

FIGS. 3A-3C show several different approaches to electrode surface modification in accordance with the present invention to allow both capture reagent (cAb) binding and achieving desired electrical properties. It should be noted that in this figure gold was taken as building material example for biosensor electrode construction. Similar, adapted surface modification strategies apply to other electrode materials, such as glassy carbon, etc.

FIG. 3A depicts the deposition of a conductive layer, such as polypyrrole, which is favorably electrochemically polymerized directly from solution onto the surface of the gold electrode. This polymerization step can be performed in the presence of the capture reagent, thereby incorporating the latter into the conductive layer deposited on the electrode's surface. The resultant modified electrode is further blocked employing common blocking agents, such as bovine serum albumin (BSA), or poly(ethylene glycol) (PEG), to reduce non-specific binding of interfering substances to the electrode's surface.

FIG. 3B depicts the classical protocol used for gold electrode modification, initiated by depositing a self-assembled monolayer (SAM) on the electrode's surface. This is preferably performed employing organic thiols, either further carrying reactive end groups (e.g., amine-reactive NHS esters of ω-carboxylated thiols) or carrying end groups that can be further chemically activated (such as COOH). Following SAM formation and subsequent chemical activation of their end groups, the capture reagent is reacted with and binds to the electrode's surface, followed by blocking un-reacted chemically-reactive end groups by common blocking agents. One preferred embodiment takes advantage of the ability to completely remove the SAM formed on the gold electrode's surface by electrochemical reduction or oxidation protocols (also known as electropolishing), thereby stripping the SAM protective layer and re-exposing the gold surface to new chemical modification. This property, combined with the relatively inert nature of the SAM when bound to the gold surface, allows electrochemically-addressable electrode manipulation. As an example, two closely-spaced gold electrodes are cleaned by protocols known in the art (e.g., piranha solution and/or oxygen plasma, followed by extensive washing and electropolishing) and reacted with organic thiol (e.g., 11-mercaptoundecanol (MUol)) to produce densely-packed MUol SAM on the surface of both electrodes. Next, one of these electrodes is subjected to electrochemical reduction at (−)1.2V for 30 sec against a reference electrode, followed by confirmation of complete SAM desorption from the surface of this electrode, typically employing either cyclic voltametry or electrochemical impedance spectroscopy techniques. The electrode array is next extensively washed, followed by re-exposure to a different organic thiol such as 11-mercaptoundecanoic acid (MUA). Since exchange between surface SAM molecules and organic thiols in solution is very slow, only the electrode from which the SAM layer has been electrochemically removed reacts with MUA, leading to the formation of MUA SAM on its surface. Further reaction of the pair of SAM-coated electrodes with N-hydroxysuccinimide (NETS) in the presence of 1-ethyl-3-(3-dimethylaminopropyl) carbodiimide (EDC) leads to the formation of an amine-reactive NHS ester exclusively on the surface of the MUA-coated electrode, but not on the MUol-coated one. Reaction of such resultant electrode pair with a capture reagent, such as an analyte-specific antibody leads to selective covalent binding and immobilization of this capture antibody on the surface of the MUA-carrying electrode, but not to the MUol-carrying one. Using similar strategies and employing multiple electrodes allows electrode-selective and exclusive SAM deposition, further allowing electrode-specific/addressable immobilization of multiple distinct analyte-specific capture reagents.

FIG. 3C depicts direct binding of the capture antibody to the nascent bare gold surface, followed by vacant electrode surface blocking. This protocol is especially useful when employing a porous electrode, characterized by high surface area that allows direct, high density reagent immobilization.

FIGS. 4A and 4B show two embodiments of the electroactive-label impedance enhancement principle for analyte detection by EIS or FET in accordance with the present invention, employing biosensor surface-immobilized, analyte-specific reagents in combination with electroactive marker-labeled soluble analyte-specific reagents. FIG. 4A depicts a typical impedance-based sensor, where the specific binding of the electroactively-labeled detection antibody-analyte complex (17-19) to the capture antibody (16) brings the electroactive label (19) in close proximity of electrode's surface (14) thereby facilitating charge transfer (20) to and from the electrode surface, which affects electrode's impedance. In the depicted example, the capture antibody (16) binding to the electrode's surface is mediated by a self-assembled monolayer (15) that creates an insulating layer between the electrode and the surrounding solution. FIG. 4B depicts a typical back-gated field effect transistor (FET), where the specific binding of the electroactively-labeled detection antibody-analyte complex (17-19) to the immobilized capture antibody (16) brings the electroactive label (19) in close proximity of the FET's semiconducting channel (25) thereby affecting channel electrical conductance due to its effect on the electric field within such channel (20). In this depicted example the current flowing from the source electrode (21) to the drain (22) through the channel (25) formed on the surface of insulator layer (23) is controlled by the gate (24) voltage and is modulated by the presence of charge carriers on the semiconducting layer surface.

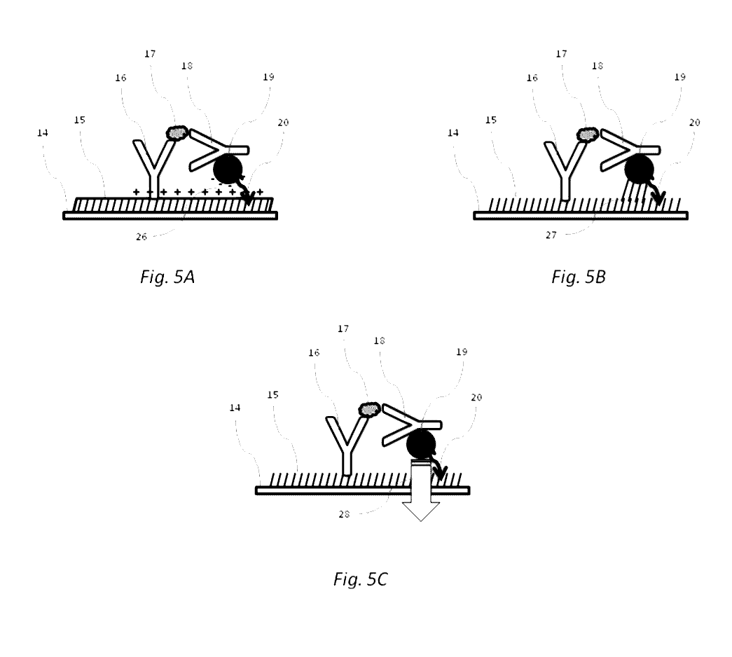

FIGS. 5A-5C depict three different embodiments of electroactive label interaction augmentation modes with the biosensor surface in accordance with the present invention. FIG. 5A illustrates an electrostatic interaction between the electroactive label (19) and the biosensor surface (14), mediated by engineering the electroactive label's zeta potential to be opposite of that of the biosensor surface (26). Such electrostatic interaction serves to both—attract the electroactive label to the biosensor surface and change the biosensor charge distribution; FIG. 5B illustrates low-affinity binding of the electroactive label (19) to the biosensor surface, mediated by engineering the electroactive label to carry chemical groups that are either chemically-complementary to or otherwise display low-affinity reactivity with ones on the biosensor surface (27). Examples for such low-affinity interactions are ones involving hydrophobic-hydrophobic forces, hydrogen bonding, dipole-dipole moments, etc. Specifically, in a preferred embodiment of the current invention, such low-affinity interaction can be tailored by the use of complementary, single-stranded DNA (cSSD) oligo pairs. In such preferred embodiment, one of the cSSD oligos is immobilized on the biosensor surface (e.g., by a 3′ or 5′ thiol modification), while its complementary strand is similarly immobilized on the electroactive label. By proper cSSD oligo pair selection, the melting temperature (Tm) of the complementary strands can be tailored to be at or below diagnostic test performance temperature (considering also the ionic strength of the surrounding buffer, among other factors) thereby fine-tuning the interaction affinity between the electroactive label and the biosensor surface. This, in-turn, allows engineering electroactive label-biosensor surface interaction forces to overcome label diffusion away from the electrode surface, while being weaker than the forces exerted by fluid flow in the lateral flow device. Such a scheme allows binding of the electroactive label to the biosensor surface only once specific interaction between the labeling reagent, analyte and capture reagent is achieved. FIG. 5C illustrates the application of an electromagnetic field (28) to attract a magnetic bead to the biosensor surface. Such magnetic beads are comprised of ferromagnetic or paramagnetic particles that are conjugated to the labeling reagent and which are further modified by coating their surface with appropriate electroactive materials. One such magnetic bead surface modification protocol involves attaching polyelectrolyte molecules to the magnetic bead surface. An additional example is employing noble metal-coated magnetic beads as an electroactive label, thereby providing a conductive surface allowing charge transfer between the electrode and the magnetic bead. In this example, the electromagnetic field is applied to the biosensor only after specific binding of the electroactive label-analyte complex to the biosensor-immobilized capture reagent has been achieved, thereby attracting only the specifically-bound electroactive label to the biosensor surface.

It should be re-emphasized that proper selection and construction of the electroactive label vis-à-vis the biosensor surface are required in order to prevent non-specific interaction between the two, promoting such interaction only upon the specific interaction between the electroactively-labeled (19) detection reagent (18), the analyte (17) and the capture reagent (16). This is achieved by balancing such low-affinity interaction forces against the lateral flow forces that act to drive the electroactive label away from the surface of the biosensor electrode. Such delicate balance assures that lateral flow forces overcome these low-affinity interaction forces in the absence of specific binding of the analyte electroactive-label complex to the capture reagent.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A biological sample device for detecting the presence or absence of specific analytes in a sample, comprising:
a porous sample pad configured to collect a sample, wherein the sample is at least one of a clinical sample and a pre-processed sample;
a first membrane in liquid communication with the sample pad, the first membrane being configured to receive from the sample pad an analyte and including an analyte-specific, electroactively-labeled detection reagent containing an electroactive label;
a second membrane in liquid communication with the first membrane, the second membrane being configured to receive from the first membrane an analyte electroactive label complex formed by interaction of the analyte with the analyte-specific, electroactively-labeled detection reagent in the first membrane;
a biosensor in liquid communication with the second membrane, the biosensor including an analyte-binding capture reagent configured to immobilize the analyte electroactive label complex on a surface of the biosensor and being configured to generate an electric signal based on direct interaction of the electroactive label component of the analyte electroactive label complex with the surface of the biosensor; and
an electronic processing unit in electrical communication with the biosensor configured to receive the electric signal from the biosensor, determine from the electrical signal whether the analyte is present in the sample, and if analyte is present in the sample to output a signal indicative of the presence of the analyte.

2. The biological sample device of claim 1, wherein the analyte-specific, electroactively-labeled detection reagent is a biomolecule having a micro-molar or higher affinity for the analyte.

3. The biological sample device of claim 2, wherein the analyte-specific, electroactively-labeled detection reagent biomolecule includes at least one of the group of an antibody, DNA, an antigen, an aptamer, an enzyme and a receptor.

4. The biological sample device of claim 1, wherein the electroactive label of the electroactively-labeled detection reagent is at least one of a molecule and a particle, and has at least one property of
being conductive,
having a dielectric constant different than a media surrounding the biosensor element,
being electrochemically reactive,
catalyzing generation of an electrochemically reactive species, and
having an electric charge.

5. The biological sample device of claim 4, wherein the molecule is a polymer and particle is a metal nanoparticle.

6. The biological sample device of claim 4, wherein the electroactive label is at least one of
electrically-conductive metal nanoparticles,
electrically-conducting carbon nanotubes, and
high-charge polyelectrolytes.

7. The biological sample device of claim 1, wherein the biosensor is amperometric, potentiometric or impedimetric, and the biosensor surface includes a sensing element carrying the analyte-binding capture reagent.

8. The biological sample device of claim 7, wherein the analyte-binding capture reagent includes at least one of an antibody, an antigen, DNA, an aptamer, an enzyme, a receptor and a molecule having micro-molar or higher affinity for the analyte.

9. The biological sample device of claim 1, wherein the biosensor surface and electroactive label are configured such that when the analyte electroactive label complex is immobilized on the surface of the biosensor via the analyte-binding capture reagent, the-bound electroactive label is in sufficiently close proximity to the sensor surface for electrical interactions between the electroactive label and the biosensor surface to generate an electrical signal detectable by the biosensor.

10. The biological sample device of claim 1, wherein the electronic processing unit communicates the electronic processing unit signal indicative of the presence of the analyte to a display unit.

11. The biological sample device of claim 10, wherein the electronic processing unit communicates the electronic processing unit signal indicative of the presence of the analyte to a display unit via wireless connection.

12. The biological sample device of claim 9, wherein the immobilization of the analyte electroactive-label complex to the biosensor surface by the capture reagent-bound includes low affinity interactions which bring the analyte electroactive label in closer proximity to the biosensor surface, the low affinity interactions including at least one of
electrostatic affinity,
chemical complementarity affinity, and
magnetic attraction.

13. The biological sample device of claim 12, wherein the low affinity interactions include at least one of
the electrostatic interaction being generated by the analyte electroactive label having a zeta potential opposite to that of the biosensor surface,
the chemical complementarity interaction being generated by at least one of hydrophobic-hydrophobic forces, hydrogen bonding, and dipole-dipole moments, and
the magnetic interaction be generated by application of an external magnetic field to attract a magnetic bead to the biosensor surface.

14. The biological sample device of claim 1, further comprising:
a third membrane located between the sample pad and the first membrane,
wherein the third membrane is configured to fractionate the biological sample passing from the sample pad to the first membrane.
